(19) United States
(12) Patent Application Publication
(10) Pub. No.: US 2025/0275531 A1
(43) Pub. Date: Sep. 4, 2025

(54) STABILIZATION MEDIUM FOR EXTENDING TEMPORAL VIABILITY OF HUMAN SEMEN

(71) Applicants: Ashwin Ramachandran, Melbourne (AU); Abdul Moiz Syed, Thornbury (AU)

(72) Inventors: Ashwin Ramachandran, Melbourne (AU); Abdul Moiz Syed, Thornbury (AU)

(21) Appl. No.: 18/593,974

(22) Filed: Mar. 3, 2024

(30) Foreign Application Priority Data
Mar. 1, 2024 (GB) GB2402973.8

(51) Int. Cl. A01N 1/02; C12N 5/076

(52) U.S. Cl. CPC A01N 1/126; A01N 1/124; C12N 5/061

(57) ABSTRACT

A semen stabilization medium is described, which includes pH buffer agents, inorganic salts, organic compounds, and amino acids with antioxidant properties. The stabilization medium preserves semen viability for up to 72 hours when the semen is mixed with the stabilization medium and maintained at a temperature within a 20% tolerance of human body temperature.

### FIELD OF THE DISCLOSURE

The present disclosure generally relates to maintaining or preserving the viability of biological materials in vitro. More particularly, the present disclosure relates to a stabilization medium and a method for extending the viability of human semen samples.

### BACKGROUND

Human semen is a complex biological substance composed of spermatozoa and seminal plasma. Semen plays a pivotal role in male fertility diagnostics and is central to various clinical and diagnostic applications, ranging from assisted reproductive technology (ART) to sperm banking and medical research. The process of sperm banking is particularly crucial for individuals at risk of genetic diseases, undergoing chemotherapy, or delaying parenthood. Semen analysis, which assesses sperm count, motility, morphology, and volume, is an essential step in evaluating male fertility health and suitability for these applications. In addition, semen analysis is used to examine the presence of sperm in a semen sample post vasectomy.

However, traditional semen analysis methods require on-site collection, posing challenges related to psychological barriers, performance anxiety, and inconvenience. Moreover, discreet options offered by clinics may be subject to time-sensitive constraints due to the perishable nature of sperm outside the male body. Cryopreservation and mail-in semen analysis kits are existing solutions, but they have limitations. Cryopreservation can negatively impact sperm quality, while mail-in kits require stabilizing semen during transport and have a limited storage duration.

Therefore, there is a clear need for a more effective medium and method for preserving human semen for long durations, free from DNA-depleting compounds like glycerol, and without the need for cryopreservation, to ensure the prolonged viability of semen samples for various purposes, including infertility diagnosis, semen donation, artificial insemination, in vitro fertilization, post vasectomy semen analysis (PVSA), and laboratory use and research.

### OBJECTIVES OF THE DISCLOSURE

The primary objective of the present disclosure is to provide an improved technical solution that overcomes the problems encountered in the prior art in storing or preserving semen samples for longer duration.

Another objective of the present disclosure is to develop a medium with a capacity to effectively preserve and stabilize human semen samples over extended time periods.

Yet another objective of the disclosure is to stretch the temporal constraints of semen sample viability, thereby broadening the logistical possibilities for the transportation and analysis of such samples.

Yet another objective of the present disclosure is to achieve an optimal dilution of semen with the medium, extending the sperm's shelf life from the typical 1-hour window to as much as 3 days.

Yet another objective of the present disclosure is to enable a method for transportation of human semen samples from the collection point to the testing centers for semen analysis.

Yet another objective of the present disclosure is to facilitate semen preservation and stabilization without a need for complex laboratory procedures like cryopreservation or centrifugation.

Additionally, the present disclosure aims to enable the preservation and stabilization of human semen samples during transportation to a laboratory without requiring refrigeration.

### SUMMARY OF THE DISCLOSURE

In order to achieve aforementioned objectives of the present disclosure, in one aspect, a stabilization medium for extending temporal viability of human semen is provided. The stabilization medium comprises one or more pH buffer agents, one or more inorganic salts, one or more organic compounds, and one or more amino acids with antioxidant properties, thereby enabling preservation of semen viability for up to a predetermined time period when the human semen is mixed with the stabilization medium and maintained within a predetermined temperature range. In some embodiments, the predetermined time period is up to 72 hours and the predetermined temperature range is 36.1 to 37.2 degree Celsius.

In some embodiments, each amino acid of the one or more amino acids in the stabilization medium has a concentration in a range of 0.01 to 0.5 millimoles (mmol).

In some embodiments, the one or more amino acids are selected from a group comprising Alanine (ala), Aspartate (asp), Asparagine (asn), Glutamate (glu), Alanyl-Glutamine (ala-gln), Glycine (gly), Proline (pro) and Serine (ser).

In some embodiments, the one or more organic compounds are selected from a group comprising glucose, sodium lactate (L-isomer), sodium pyruvate and Taurine (tau). The stabilization medium comprises Taurine (tau) in a concentration range of 0.01 to 10.0 mmol. Optionally, the stabilization medium comprises glucose having a concentration between 0.05 to 5.0 mmol. Optionally, the stabilization medium comprises sodium lactate (L-isomer) having a concentration between 5.0 to 20.0 mmol. The stabilization medium may comprise sodium pyruvate in a concentration between 0.1 to 1.0 mmol.

In some embodiments, the one or more pH buffer agents comprise (3-(N-morpholino) propanesulfonic acid) (MOPS) or 4-(2-Hydroxyethyl)-1-piperazine ethanesulfonic acid (HEPES) in a concentration range of 10.0 to 25.0 mmol.

In some embodiments, the one or more inorganic salts are selected from a group comprising sodium chloride (NaCl), potassium chloride (KCl), magnesium sulfate heptahydrate (MgSO₄7H₂O), sodium phosphate dihydrate (Na₂PO₄2H₂O), sodium bicarbonate (NaHCO₃), and calcium chloride dihydrate (CaCl₂. 2H₂O). The one or more inorganic salts may comprise CaCl₂ 2H₂O having a concentration between 0.8 to 2.8 mmol. The one or more inorganic salts may comprise MgSO₄7H₂O having a concentration between 0.2 to 4 mmol. The one or more inorganic salts may comprise sodium chloride (NaCl) in a concentration between 75.0-105 mmol. The one or more inorganic salts may comprise potassium chloride (KCl) in a concentration between 3.5-7.5 mmol. The one or more inorganic salts may comprise sodium phosphate dihydrate (Na₂PO₄2H₂O) in a concentration between 0.05-1.5 mmol. The one or more inorganic salts may comprise sodium bicarbonate (NaHCO₃) in a concentration between 2.0-10.0 mmol.

In some embodiments, the stabilization medium further comprises one or more antioxidants. The one or more antioxidants may be selected from a group comprising Acetyl-L-Carnitine, Alpha-Lipoic Acid, and N-Acetyl-L-Cysteine. Optionally, the stabilization medium further comprises one or more pH-altering compounds. The one or more pH-altering compounds may be selected from a group comprising sodium dihydrogen phosphate dihydrate (NaH₂PO₄2H₂O), magnesium sulfate heptahydrate (MgSO₄7H₂O), calcium chloride dihydrate (CaCl₂2H₂O), sodium lactate (L-isomer) and sodium pyruvate. Optionally, the stabilization medium further comprises one or more antibiotics. Optionally, the stabilization medium further comprises chelating agents.

In some embodiments, the stabilization medium has a pH between 7.2 and 8.

In another aspect, a method for extending temporal viability of human semen is provided. The method comprises mixing the human semen with a stabilization medium and maintaining a mixture of the human semen and the stabilization medium within a temperature range of 36.1 to 37.2 degree Celsius, thereby enabling preservation of semen viability for an extended period of up to 72 hours.

In yet another aspect, a kit comprising a stabilization medium is provided. The stabilization medium comprises one or more pH buffer agents, one or more inorganic salts, one or more organic compounds, and one or more amino acids with antioxidant properties, thereby enabling preservation of semen viability for an extended period up 72 hours when the human semen is mixed with the stabilization medium and maintained within a temperature range of 36.1 to 37.2 degree Celsius.

The stabilization medium of the present disclosure effectively preserves and stabilizes the human semen samples over extended time periods specifically up to 72 hours without any refrigeration or cryopreservation. The stabilization medium extends the sperm's shelf life from the typical 1-hour window to as much as 3 days. Accordingly, the stabilization medium broadening logistic possibilities for the transportation of semen samples and ensures the prolonged viability of semen samples for various purposes, including infertility diagnosis, semen donation, artificial insemination, in vitro fertilization, post vasectomy semen analysis (PVSA), and other laboratory use and research. Further, the stabilization medium is free of DNA-depleting compounds like glycerol, thereby eliminating damage to the sperm's genetic material, improves sperm viability, leads to higher fertilization rate, minimize the risk of genetic abnormalities in the preserved semen cells, and may improve reproductive outcome.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The essence and intricacies of the current invention can be further elucidated through a detailed examination of the accompanying figures, which serve to illuminate the various objectives, features, components, and advantages to those who possess expertise in this field. These figures, when considered in conjunction with the text of this patent application, provide a holistic understanding of the invention's aims, functionalities, and advantages. They serve as a valuable resource for translating the invention's technical aspects into actionable steps that can be effortlessly understood and implemented by professionals in the field.

FIGS. 1-2 are tables that illustrate a composition of a stabilization medium in accordance with the present disclosure.

FIGS. 3-4 are graphical illustrations of an ability of a stabilization medium in preserving progressive motility parameter of semen samples over an extended period in accordance with the present disclosure.

FIGS. 5-6 are graphical illustrations of an ability of a stabilization medium in preserving non-progressive motility parameter of semen samples over an extended period in accordance with the present disclosure.

FIGS. 7-8 are graphical illustrations of an ability of a stabilization medium in preserving immotile parameter of semen samples over an extended period in accordance with the present disclosure.

FIGS. 9-10 are graphical illustrations of an ability of a stabilization medium in preserving morphology parameter of semen samples over an extended period in accordance with the present disclosure.

FIG. 11 is a graphical illustration comparing progressive motility parameter of neat human semen samples against the semen samples mixed with a stabilization medium at T=0, 24, 48, and 72 hours in accordance with the present disclosure.

FIG. 12 is a graphical illustration comparing non-progressive motility parameter of neat human semen samples against the semen samples mixed with a stabilization medium at T=0, 24, 48, and 72 hours in accordance with the present disclosure.

FIG. 13 is a graphical illustration comparing morphology parameter of neat human semen samples against the semen samples mixed with a stabilization medium at T=0, 24, 48, and 72 hours in accordance with the present disclosure.

FIG. 14 is a graphical illustration comparing immotile parameter of neat human semen samples against the semen samples mixed with a stabilization medium at T=0, 24, 48, and 72 hours in accordance with the present disclosure.

FIG. 15 is a graphical illustration comparing total motility parameter of neat human semen samples against the semen samples mixed with a stabilization medium at T=0, 24, 48, and 72 hours in accordance with the present disclosure.

FIG. 16 is a table illustrates DNA fragmentation results of neat human semen samples and the semen samples mixed with a stabilization medium at T=0 and 72 hours in accordance with the present disclosure.

FIG. 17 is a block diagram illustrating a kit encompassing a stabilization medium in accordance with the present disclosure.

FIG. 18 is a flowchart that illustrates a method of stabilization of a human semen sample in accordance with the present disclosure.

### DETAILED DESCRIPTION

The following description describes various features and functions of the disclosed stabilized medium with reference to the accompanying figures. The terminology used herein is for the purpose of describing particular embodiments only and is not intended to limit the scope of the present invention.

The detailed description is construed as a description of the currently preferred embodiments of the present invention and does not represent the only form in which the present invention may be practiced. This is to be understood that the same or equivalent functions may be accomplished, in any order unless expressly and necessarily limited to a particular order, by different embodiments that are intended to be encompassed within the scope of the present invention.

The embodiment is chosen and described to provide the best illustration of the principles of the invention and its practical application and to enable one of ordinary skill in the art to utilize the invention in various embodiments and with various modifications as are suited to the particular use contemplated.

Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

As mentioned, there remains a need for an improved technical solution that overcomes a problem of preserving semen sample over an extended period. The present disclosure provides a stabilization medium and a method for stabilizing human semen sample over an extended period without cryopreservation and refrigeration. The stabilization medium of the present disclosure is advantageous which effectively preserves and stabilizes the human semen samples over extended time periods specifically up to 72 hours without any refrigeration or cryopreservation. The stabilization medium extends the sperm's shelf life from the typical 1-hour window to as much as 3 days. Consequently, the stabilization medium widens logistic possibilities for the transportation of semen samples and ensures the prolonged viability of semen samples for diverse applications, including infertility diagnosis, semen donation, artificial insemination, in vitro fertilization, post vasectomy semen analysis (PVSA), and other laboratory use and research. Further, the stabilization medium is free of DNA-depleting compounds like glycerol, thereby eliminating damage to the sperm's genetic material. Such that, the stabilization medium improves sperm viability, leads to higher fertilization rate, minimize the risk of genetic abnormalities in the preserved semen cells, and may improve reproductive outcome.

As used herein, several terms are defined below:

The term “semen sample” as used herein, generally refers to an organic bodily fluid secreted by the male gonads and other sexual organs of male or hermaphroditic animals and can fertilize the female ovum.

The terms “stabilization medium” refers to a substance or a mixture of substances a solution or a material designed to maintain the integrity, viability, or stability of biological specimens or materials during storage or transportation. It is used to prevent degradation, damage, or alteration of the biological components, ensuring that they remain in a suitable condition for subsequent analysis or processing. The biological specimens or materials may be a semen sample.

The term “temporal viability” as used herein, generally refers to an ability of a biological material to remain alive, functional, or viable over a specific period of time. It is a measure of how well an organism, cell, or biological material can maintain its integrity and functionality over a given temporal duration.

The term “extended temporal viability” as used herein, generally refers to an ability of a biological material to remain alive, functional, or viable over a prolonged period of time.

The term “preservation” as used herein, generally refers to an act or process of maintaining the quality, condition, or existence of a substance or a solution or a material over time.

The term “transporting” as used herein, generally refers to an act or process of movement or conveyance of collected specimens, materials, or substances from one location to another location.

The term “semen viability” as used herein, generally defined as a percentage of live sperm found in a semen sample.

The term “pH buffer agent” as used herein, generally refers to a substance or a mixture of substances that helps to maintain a relatively constant pH (acidity or alkalinity) in a solution, even when an acid or a base is added.

The term “inorganic salts” as used herein, generally refers to a chemical compound consisting of an ionic assembly of positively charged cations and negatively charged anions, which results in a compound with no net electric charge.

The term “organic compound” as used herein, generally refers to a chemical compound in which one or more atoms of carbon are covalently linked to atoms of other elements.

The term “amino acids with antioxidant properties” as used herein, generally refers to amino acids that possess the ability to neutralize or reduce oxidative stress and prevent the damaging effects of free radicals within a biological or chemical system.

The term “antioxidants” as used herein, generally refers to a substance or a mixture of substances that scavenge and neutralize free radicals or reactive oxygen species. Reactive oxygen species are highly reactive molecules or ions that contain oxygen and can cause cellular damage by initiating oxidative stress. Antioxidants play a crucial role in maintaining cellular health by preventing or slowing down the oxidative damage caused by free radicals.

The term “pH-altering compounds” generally refers to a substance or a mixture of substances that have the ability to change the acidity or alkalinity (pH level) of a solution.

The term “antibiotics” generally refers to a substance or a mixture of substances that destroy bacterial cells or inhibit growth of the bacterial cells.

The term “chelating agent” generally refers to a substance or a mixture of substances that form a complex with a metal ion by binding to it through multiple coordination sites.

The term “pH” generally refers to a measure of the acidity or alkalinity of a solution.

The term “concentration” generally refers to a quantity or an amount of a substance contained within a particular volume or mass of a mixture. The concentration of a solution is typically expressed in terms of mass, volume, or the number of moles.

The term “predetermined time period” generally refers to a specific and known amount of a time set in advance for a particular purpose.

The term “predetermined temperature range” generally refers to a specific and known amount of a temperature set in advance for a particular purpose.

The term “one or more” as used herein, generally refers to at least one and also more than one.

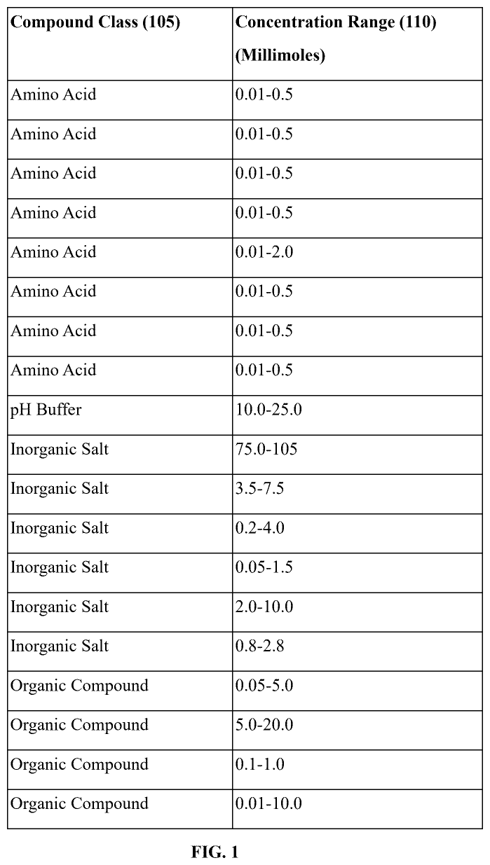

FIG. 1 is a table 100 that illustrates a composition of a stabilization medium, in accordance with the present disclosure. The table 100 shows compound class 105 and concentration range 110 in the stabilization medium. The stabilization medium is a buffer medium that preserves human semen thereby enabling human semen storage and transportation to labs, etc. for analysis and for diverse applications. The diverse applications of human semen include, but not limited to, semen donation, artificial insemination, in vitro fertilization, and other applications. The stabilization medium comprises at least four major compound classes 105 namely amino acids, pH buffer agents, inorganic salts, and organic compounds.

In some embodiments, amino acids that includes, but not limited to, alanine, aspartate, asparagine, glutamate, alanyl-glutamine, glycine, proline, serine, and taurine are added in the stabilization medium for their antioxidant properties. The amino acids with antioxidant properties shield sperm cells from cold shock and contribute to epigenetic homeostasis. Antioxidants are vital in mitigating oxidative stress, which, if left unaddressed, can result in sperm pathology, including adenosine triphosphate (ATP) depletion, inadequate axonemal phosphorylation, lipid peroxidation, and loss of motility and viability. Moreover, amino acids and antioxidants play a pivotal role in safeguarding sperm cells from the detrimental effects of reactive oxygen species (ROS). Failure to manage ROS can result in degradation and harm to the sperm sample, ultimately undermining the stabilization medium's effectiveness. In some embodiments, the amino acids include one or more of alanine, aspartate, asparagine, glutamate, alanyl-glutamine, glycine, proline, and serine.

The pH buffer agents of the stabilization medium may include 3-(N-morpholino) propanesulfonic acid (MOPS) and 4-(2-Hydroxyethyl)-1-piperazine ethanesulfonic acid (HEPES). The pH buffer agents help in maintaining the pH that is crucial for sperm motility. The inorganic salts of the stabilization medium may be selected from a group comprising sodium chloride (NaCl), potassium chloride (KCl), magnesium sulfate heptahydrate (MgSO₄7H₂O), sodium phosphate dihydrate (Na₂PO₄2H₂O), sodium bicarbonate (NaHCO₃), and calcium chloride dihydrate (CaCl₂ 2H₂O). The inorganic salts play a crucial role in maintaining the integrity and viability of sperm cells during storage. For example, primary functions of inorganic salts in stabilization media may include osmotic regulation, ion balance, pH buffering, preventing ice crystal formation, and energy metabolism. The organic compounds of the stabilization medium may be selected from a group comprising glucose, sodium lactate (L-isomer), sodium pyruvate, Taurine (tau). The organic compounds play a crucial role in enhance cell viability and protect against damage during storage. For example, primary functions of organic compounds in stabilization media may include membrane stabilization, osmotic regulation, energy source, mimicking physiological environment, viscosity adjustment, and nutrient supply. In some embodiments, the stabilization medium may include antibiotics such as gentamicin or penicillin. The antibiotics serves the critical purpose of preventing microbial contamination within the semen sample. The antibiotics maintain the sample's sterility, ensuring that the semen sample remains free from harmful microorganisms. The usage of antibiotics must strike a balance. Overuse can harm sperm cells, while inadequate usage may leave the sample vulnerable to microbial contamination. In some embodiments, the stabilization medium includes chelating agent such as EDTA that aids in extending the viability of the semen sample by stimulating the motility of the sperm cells. On one hand excessive EDTA may damage sperm cells, while, on the other hand, insufficient EDTA may fail to stimulate sperm motility, thus affecting the preservation of sperm viability. In some embodiments, the stabilization medium includes one or more pH-altering compounds that may be selected from a group comprising sodium dihydrogen phosphate dihydrate (NaH₂PO₄2H₂O), magnesium sulfate heptahydrate (MgSO₄7H₂O), calcium chloride dihydrate (CaCl₂2H₂O), sodium lactate (L-isomer) and sodium pyruvate. In some embodiments, the stabilization medium includes one or more antioxidants that may be selected from a group comprising Acetyl-L-Carnitine, Alpha-Lipoic Acid, and N-Acetyl-L-Cysteine.

In some embodiments, the ingredients of the stabilization medium such as the amino acids, pH buffer agents, inorganic salts, and organic compounds are dissolved in water to form the stabilization medium.

The combination of compound classes 105 of table 100 enables human semen to be preserved for up to 72 hours, unlike conventions or existing mediums that allow preservations up to 1 hour. In some embodiments, the stabilization medium may be refrigerated with an icepack in order to maintain the temperature of the stabilization medium between 2 and 12 degree Celsius before human semen is mixed with the stabilization medium forming a mixture. It is to be noted that the stabilization medium needs to be brought to room temperature before adding the human semen sample into the stabilization medium to ensure the desired effect of stabilization. The mixing involves agitating the mixture comprising the human semen sample and the stabilization medium sufficiently to make the mixture homogeneous. Post the mixing, the mixture is maintained closer to human body temperature. In one exemplary embodiment, the mixture of human semen sample and the stabilization medium is maintained between 36.1 to 37.2 degree Celsius. In another exemplary embodiment, the mixture of human semen sample and the stabilization medium is maintained at a temperature within a 20% tolerance of human body temperature. In some embodiments, the stabilization medium has a pH between 7.2 and 8.

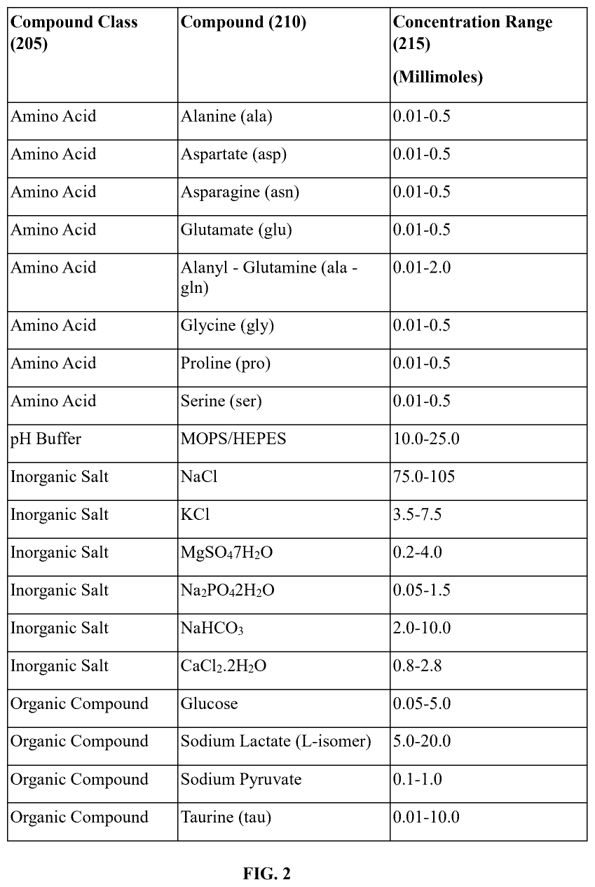

FIG. 2 is a table 200 illustrates a composition of a stabilization medium in accordance with the present disclosure. The table 200 includes a compound class 205, a compound 210 and a concentration range 220 in the stabilization medium. The compound class 205 includes amino acids, pH buffer agents, inorganic salts and organic compounds. The amino acids may be, but not limited to, Alanine (ala), Aspartate (asp), Asparagine (asn), Glutamate (glu), Alanyl-Glutamine (ala-gln), Glycine (gly), Proline (pro), and Serine (ser). The stabilization medium comprises each amino acid in a concentration of 0.01 to 0.5 millimoles (mmol). It's crucial to maintain the concentration of the amino acids, in the stabilization medium, within a provided or given range. Exceeding the given range of the amino acids may be led to an excess of antioxidants in the stabilization medium which can damage sperm cells, diminishing the function of the sperm cells, while falling short of given range of the amino acids may reduce the stabilization medium's ability to shield sperm cells from oxidative stress. In some embodiments, the stabilization medium may include antioxidants such as, but not limited to, Acetyl-L-Carnitine, Alpha-Lipoic Acid, and N-Acetyl-L-Cysteine, to protect the sample from oxidative stress in the stabilization medium.

The stabilization medium further includes pH buffer agents such as, but not limited to, MOPS or HEPES having a concentration range of 10.0 to 25.0 mmol. MOPS and HEPES, are designed to maintain an optimal pH range for sperm, i.e., between 7.2 and 8. Excessive MOPS can render the stabilization medium overly acidic, thereby failing to ensure the stabilization medium's pH remains within the optimal range. Conversely, too little or lower amount of MOPS/HEPES may reduce the buffer capacity, hampering the stabilization medium's ability to effectively maintain the pH, which is crucial for sperm motility and function. In some embodiments, the stabilization medium includes pH-Altering compounds such as NaH₂PO₄2H₂O that may act as an acid or base, MgSO₄7H₂O and CaCl₂2H₂O that may be neutral to slightly acidic effect on pH, and sodium lactate (L-isomer) and sodium pyruvate that can serve as acids or bases depending on the specific circumstances.

The stabilization medium further includes inorganic compounds such as, but not limited to, NaCl, KCl, MgSO₄7H₂O, Na₂PO₄2H₂O, NaHCO₃ and CaCl₂2H₂O having concentration of 75.0 to 105 mmol, 3.5 to 7.5 mmol, 0.2 to 4.0 mmol, 0.05 to 1.5 mmol, 2.0 to 10.0 mmol and 0.8 to 2.8 mmol respectively. Inclusion of inorganic compounds helps maintain the ideal osmotic balance in the semen sample, which is vital for sperm cell integrity and function. NaCl and KCl supply cations to the semen sample, which are indispensable for maintaining the osmotic balance. Furthermore, Na+, K+, and NaHCO₃ contribute to maintaining the intracellular pH of the semen sample. Too few cations from NaCl and KCl can disrupt the balance of osmotic pressure, which is paramount for preserving sperm integrity and function, while an excess can led to hyperosmotic conditions, both negatively impacting sperm. Similar challenges may arise with human serum albumin (HAS), which also contributes to osmotic pressure regulation. Further, MgSO₄7H₂O provides magnesium ions (Mg2+) which plays a role in various cellular functions and is involved in the stabilization of nucleic acids, thereby contributing to the overall stability of sperm cells. Na₂PO₄ 2H₂O supplies sodium ions (Na+) and phosphate ions (PO₄³⁻) which maintains the osmotic balance and helps to regulate pH, thereby contributing to the stability of the intracellular environment of sperm. CaCl₂ 2H₂O provides calcium ions (Ca²⁺) which is essential for various cellular processes, including sperm motility, thereby contributing to the overall stability and function of sperm cells. The combined action of one or more inorganic salts in the stabilization medium according to the present disclosure helps in creating an optimal environment for preserving sperm viability, maintaining osmotic balance, and supporting essential cellular functions. The specified concentration ranges ensure that the one or more inorganic salts play their roles effectively without causing detrimental effects on sperm health.

Moreover, the stabilization medium further includes organic compounds such as, but not limited to, glucose, sodium lactate (L-isomer), sodium pyruvate and Taurine (tau) having concentration ranges of 0.05 to 5.0 mmol, 5.0 to 20.0 mmol, 0.1 to 1.0 mmol and 0.01 to 10.0 mmol respectively. The stabilization medium that has sodium pyruvate have shown to better preserve the DNA integrity of sperm samples. Addition of glucose in the stabilization medium plays a vital role in preserving the motion characteristics of sperm cells. The presence of glucose in the stabilization medium significantly impacts the motion characteristics of sperm. Inclusion of glucose is essential for preserving and promoting the desired motion characteristics of sperm. Excessive glucose can lead to unintended metabolic changes that negatively impact sperm function, while an insufficient amount may fail to adequately maintain these characteristics. Further, presence of sodium lactate contributes to the maintenance of pH and functions an energy substrate to sperm cells. Furthermore, Taurine acts as an antioxidant, helping to protect sperm cells from oxidative stress. Taurine may also contribute to maintaining osmotic balance within the sperm cells. Each of the organic compounds in the stabilization medium plays a crucial role in creating an environment that supports the health and functionality of sperm cells during preservation. The one or more organic compounds in the stabilization medium contribute to energy metabolism, help regulate pH, and provide protection against oxidative damage. The specified concentration ranges ensure that the organic compounds are present in amounts that are beneficial for sperm viability without causing any adverse effects. The combination of both inorganic and organic compounds in the stabilization medium aims to create an optimal and supportive environment for sperm cells during storage and handling.

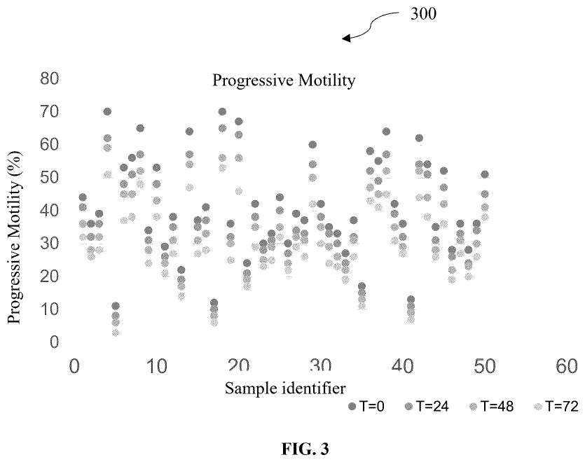

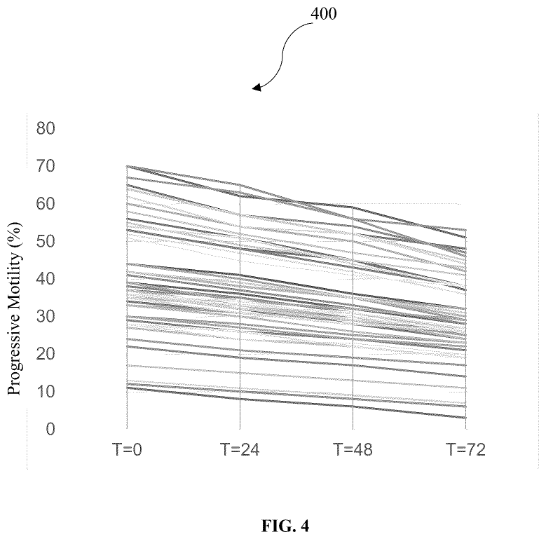

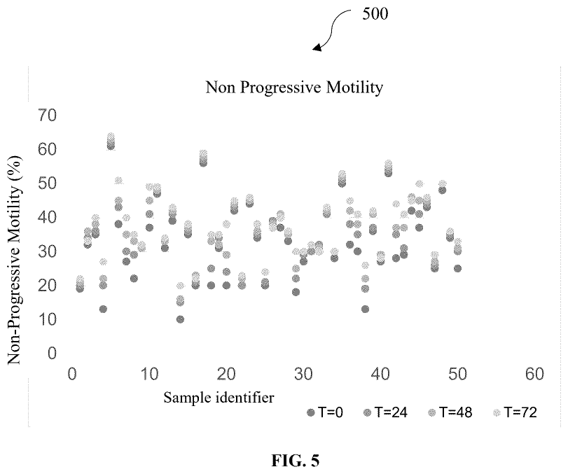

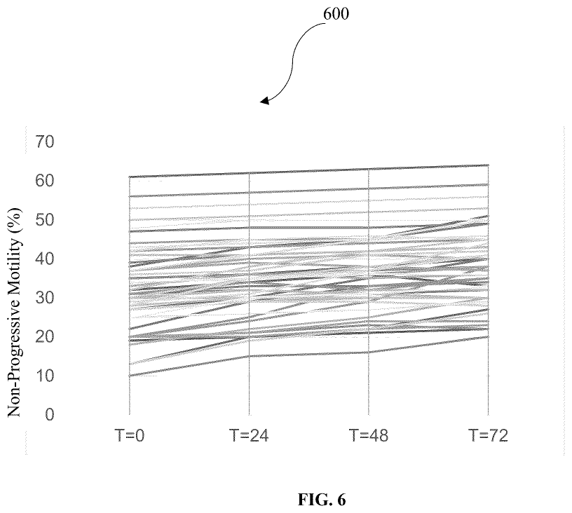

FIGS. 3-10 are graphical illustration showing experimental data from clinical trials during validation studies, in accordance with the present disclosure. The graphical illustration shows the test studies performed at 37° C., whereby neat human semen samples were mixed with the stabilization medium were tested at T=0, T=24H, T=48H, and T=72H to gather data on the performance of the stabilization medium. In particular, FIGS. 3-4 are graphical illustrations 300 and 400 of an ability of a stabilization medium in preserving progressive motility parameter of semen sample over an extended period in accordance with the present disclosure. As seen in FIGS. 3-4, progressive motility parameter of the semen sample is preserved up to 72 hours which highlights the effectiveness of the stabilization medium in maintaining the functional integrity of sperm cells over the extended period up to 72 hours. Progressive motility is a crucial factor in the assessment of sperm quality and their ability in successful fertilization. This extended preservation period is particularly significant in the context of various reproductive procedures, such as artificial insemination and in vitro fertilization, where flexibility in timing is essential. FIGS. 5-6 are graphical illustrations 500 and 600 of an ability of a stabilization medium in preserving non-progressive motility parameter of semen sample over an extended period in accordance with the present disclosure. As seen in FIGS. 5-6, non-progressive motility parameter of the semen sample is preserved up to 72 hours which underscores the efficacy of the stabilization medium in maintaining the viability and quality of sperm cells over an extended period. Non-progressive motility is an essential aspect of sperm assessment, representing the proportion of sperm that exhibit movement but lack the typical forward progression observed in progressive motility. The depicted data in FIGS. 5-6 illustrates the trends and patterns of non-progressive motility over the specified timeframe such as T=0, T=24H, T=48H, and T=72H. Understanding the dynamics of non-progressive motility preservation is crucial as it complements the assessment of overall sperm health. The prolonged preservation observed in FIGS. 5-6 suggests that the stabilization medium is adept at mitigating factors that may otherwise compromise non-progressive motility, such as environmental stressors or natural physiological changes over time.

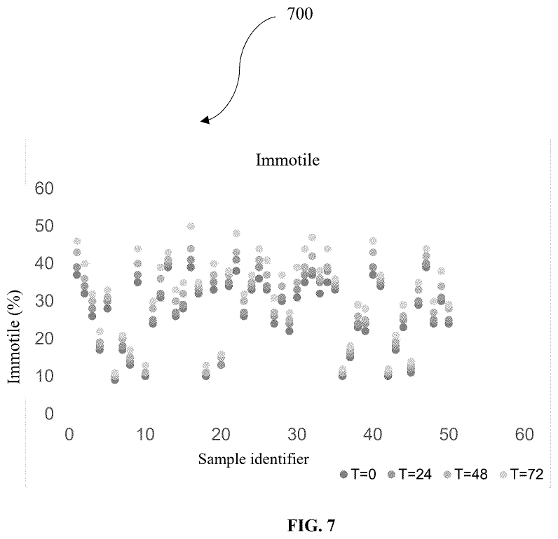

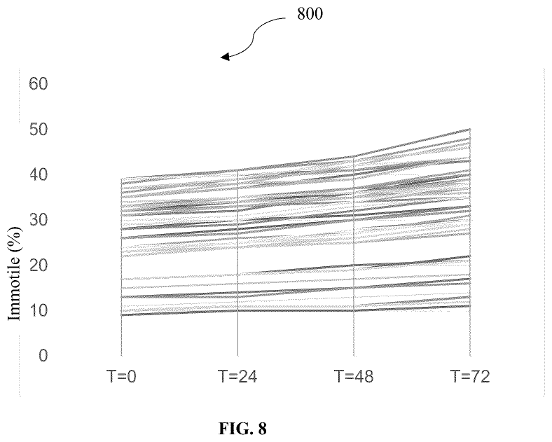

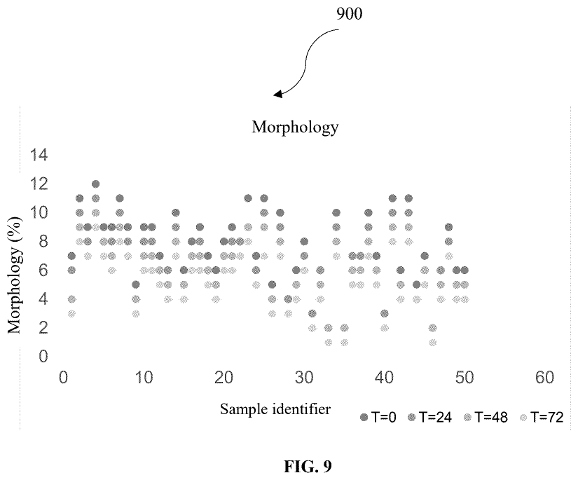

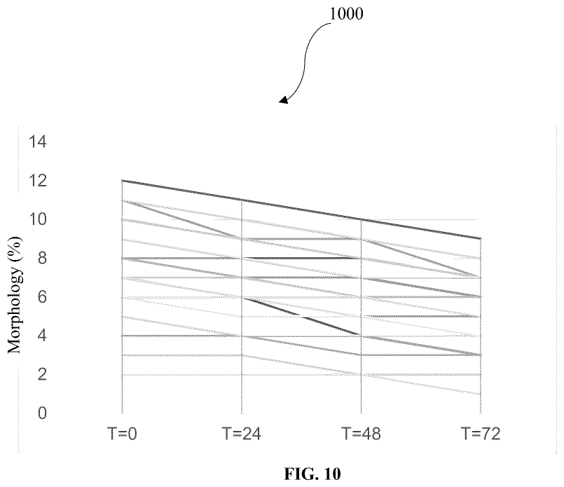

FIGS. 7-8 are graphical illustrations 700 and 800 of an ability of a stabilization medium in preserving immotile parameter of semen samples over an extended period in accordance with the present disclosure. As shown in FIGS. 7-8, the stabilization medium maintains the viable sperm cells up to 72 hours, thereby the immotile parameter of semen sample is kept constant up to 72 hours. The constancy in the immotile parameter up to 72 hours timeframe provides evidence that the stabilization medium's ability to mitigate factors that may otherwise compromise sperm viability. As a result, the preservation of viable sperm cells and the constancy of the immotile parameter demonstrated in FIGS. 7-8 underscore the potential of the stabilization medium to play a pivotal role in storing and preserving the human semen over the extended period without cryopreservation and/or refrigeration. FIGS. 9-10 are graphical illustrations 900 and 1000 of an ability of a stabilization medium in preserving morphology parameter of semen samples over an extended period in accordance with the present disclosure. FIGS. 9-10 visually emphasize the consistent maintenance of normal sperm morphology, reinforcing the stability and efficacy of the stabilization medium during the specified timeframe. The ability of the stabilization medium to uphold normal morphology up to 72 hours timeframe provides evidence that the stabilization medium's ability to preserve the semen sample. Maintaining a consistent level of normal sperm morphology over an extended period is particularly valuable in the context of assisted reproductive technologies.

It is clear from the FIGS. 3-10, the formulation of the stabilization medium effectively preserves human semen for long durations of up to 72 hours compared to 1 hour in the existing mediums. The stabilization medium is free from DNA-depleting compounds like glycerol. Moreover, the stabilization medium doesn't require cryopreservation.

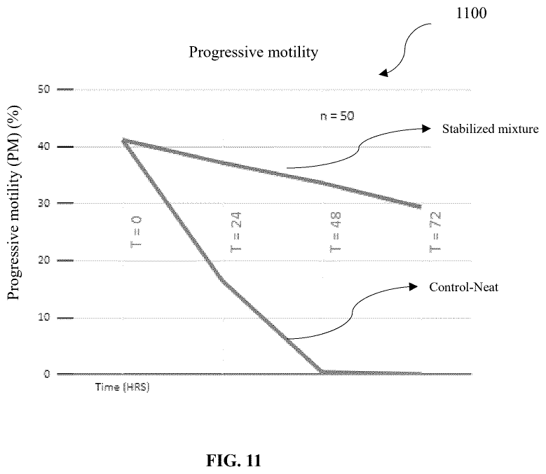

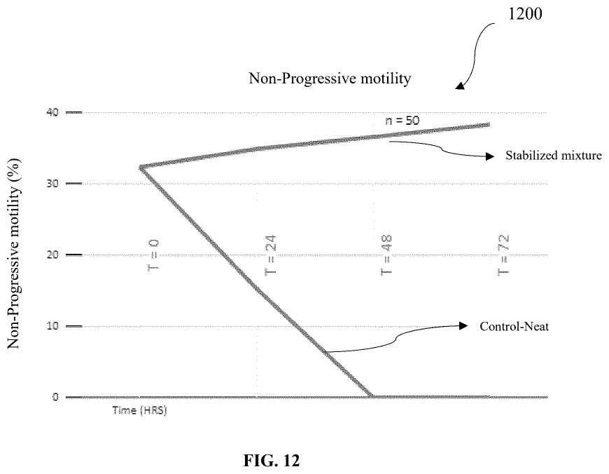

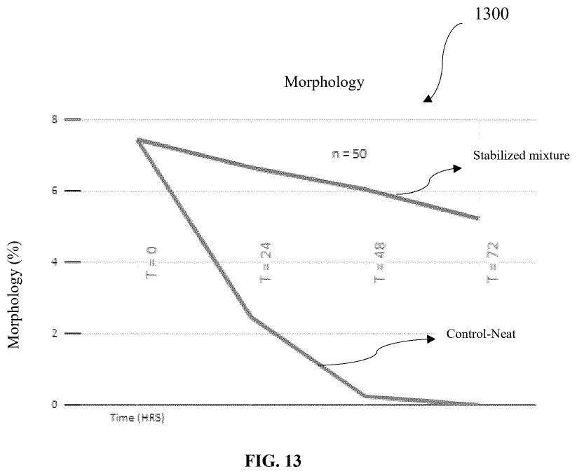

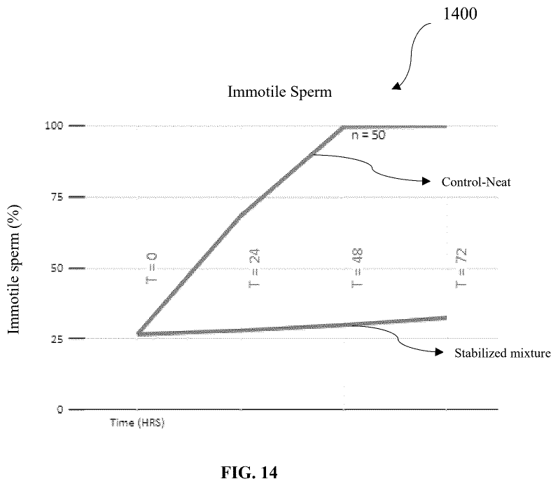

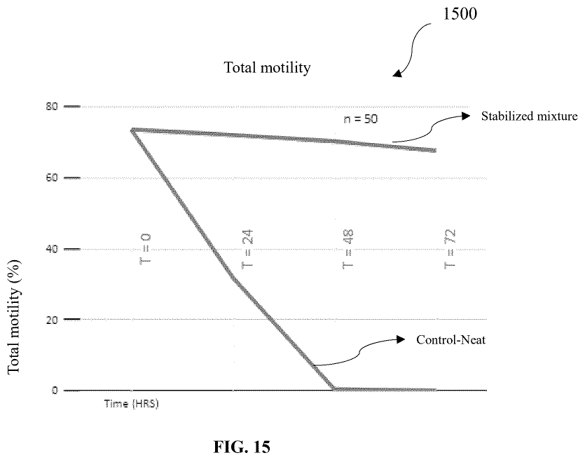

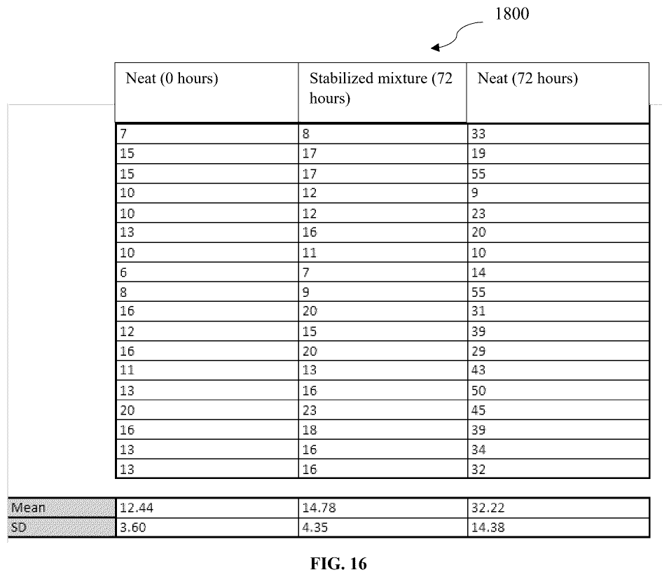

Now the neat human semen samples and the semen samples mixed with the stabilization medium were tested at T=0, 24, 48, and 72 hours for measuring progressive motility, non-progressive motility, morphology, immotile, and total motility parameters. 50 neat human semen samples were collected. The neat human semen sample is taken as a control. The human semen sample mixed with the stabilization medium in 1:3 ratio to form a stabilized mixture (i.e., 1 part semen sample and 3 parts stabilization medium). 1 ml of the stabilized mixture is tested at T=0, 24, 48, and 72 hours. The neat human semen sample is also tested at T-0, 24, 48, and 72 hours for comparison. The tests were performed at room temperature. FIG. 11 is a graphical illustration 1100 comparing progressive motility parameter of neat human semen samples against the semen samples mixed with the stabilization medium at T-0, 24, 48, and 72 hours in accordance with the present disclosure. It is to be noted that each graph was generated based on the average values of the respective parameters collected from a sample size of 50. This means that the data presented in the graphs represent the collective average measurements derived from 50 individual samples. As depicted in FIG. 11, there was a notable decrease in the percentage of progressively motile sperm in the untreated human semen samples (control) after several hours, nearly diminishing entirely by the 72-hour mark. Further, in the stabilized mixture (semen sample mixed with the stabilization medium), the percentage of progressively motile sperm were preserved for up to 72 hours. FIG. 12 is a graphical illustration 1200 comparing non-progressive motility parameter of neat human semen samples against the semen samples mixed with the stabilization medium at T=0, 24, 48, and 72 hours in accordance with the present disclosure. As seen in FIG. 12, a significant reduction in the percentage of non-progressively motile sperm was observed in the untreated human semen samples (control) after several hours, nearly reaching complete absence by the 72-hour mark. Additionally, within the stabilized mixture (semen samples mixed with the stabilization medium), the percentage of non-progressively motile sperm remained intact for the duration of 72 hours. FIG. 13 is a graphical illustration 1300 comparing morphology parameter of neat human semen samples against the semen samples mixed with a stabilization medium at T=0, 24, 48, and 72 hours in accordance with the present disclosure. As seen in FIG. 13, there was a substantial decrease in the percentage of sperm morphology observed in the untreated human semen samples (control) after several hours, nearly diminishing entirely by the 72-hour mark. Moreover, within the stabilized mixture (semen samples mixed with the stabilization medium), the percentage of sperm morphology remained preserved for the entire 72-hour duration. FIG. 14 is a graphical illustration 1400 comparing immotile parameter of neat human semen samples against the semen samples mixed with a stabilization medium at T=0, 24, 48, and 72 hours in accordance with the present disclosure. a notable rise in the percentage of immotile sperm was evident in the untreated human semen samples (control) after several hours, nearly tapering off entirely by the 72-hour mark. Conversely, within the stabilized mixture (semen samples mixed with the stabilization medium), there was no significant increase in the percentage of immotile sperm observed up to 72 hours. FIG. 15 is a graphical illustration 1500 comparing total motility parameter of neat human semen samples against the semen samples mixed with a stabilization medium at T=0, 24, 48, and 72 hours in accordance with the present disclosure. As depicted in FIG. 15, a significant decline in the percentage of total motility of sperm cells was noted in the untreated human semen samples (control) after several hours, nearly reaching complete absence by the 72-hour mark. Furthermore, within the stabilized mixture (semen samples mixed with the stabilization medium), the percentage of total motility of sperm cells remained intact for the entire 72-hour duration. FIG. 16 is a table 1600 that illustrates DNA fragmentation results of neat human semen samples and the semen samples mixed with the stabilization medium at T=0 and 72 hours in accordance with the present disclosure. As shown in the table 1600, the DNA fragmentation percentage was notably elevated at 72 hours in the untreated semen samples (control). Conversely, within the stabilized mixture, the DNA integrity of semen samples was preserved for the entire 72-hour duration.

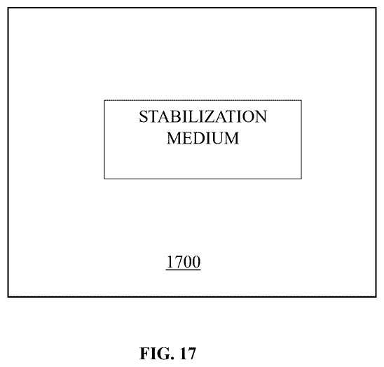

FIG. 17 is a block diagram illustrating a kit 1700 encompassing a stabilization medium in accordance with the present disclosure. The kit 1700 comprises the stabilization medium as illustrated in table 100 or table 200. The stabilization medium may be refrigerated with an icepack or any other suitable elements in order to maintain the temperature of the stabilization medium between 2 degree Celsius and 12 degree Celsius before the human semen is mixed with the stabilization medium forming a mixture. It is to be noted the stabilization medium needs to be brought to room temperature before adding the semen sample into the stabilization medium to ensure the desired effect of stabilization. In some embodiments, the stabilization medium may be stored in a container or vial and placed in a housing of the kit 1700. In some embodiments, the kit 1700 may further comprise, but not limited to, an instruction manual, any other containers, and transfer devices, temperature regulating or controlling elements. The kit 1700 may comprise an outer packaging for housing containers, the instruction manual, transfer devices, temperature regulating or controlling elements and the like.

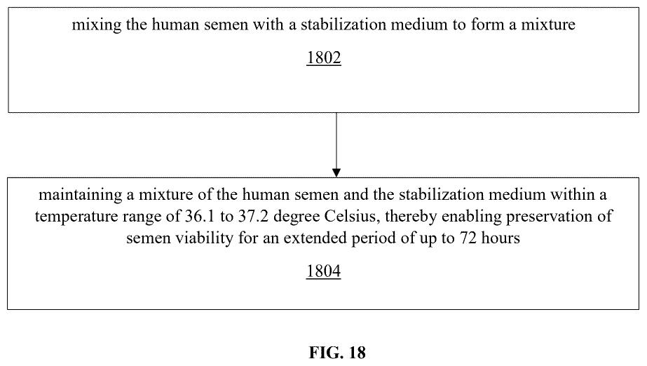

FIG. 18 is a flowchart illustrates a method of stabilization of a human semen sample in accordance with the present disclosure. At step 1802, the human semen sample is mixed with the stabilization medium of table 100 or table 200 to form a mixture. In some embodiments, 3 parts of stabilization medium is mixed with 1 part of the human semen sample. In some embodiments, the mixture of the human semen and the stabilization medium is agitated to a form a homogenous mixture or solution. In some embodiments, the stabilization medium 120 is stored or maintained between 2 degrees Celsius (° C.) and 12° C. through a use of a fridge, a gel ice pack, any other cooling elements or devices and the like, prior to mixing with the human semen sample. It is to be noted that before the stabilization medium is mixed with the human semen sample, the stabilization medium is brought to room temperature, thereafter the stabilization medium is mixed with the human semen sample to ensure the desired effect of stabilizing the human semen sample. At step 1804, the mixture of the human semen and the stabilization medium is maintained within a temperature range of 36.1 to 37.2 degree Celsius, thereby enabling preservation of semen viability for an extended period of up to 72 hours. In some embodiments, the mixture of human semen sample and the stabilization medium is maintained at a temperature within a 20% tolerance of human body temperature.

It is to be understood that while we have illustrated and described certain forms of the present invention, it is not to be limited to the specific forms or arrangement of parts herein described and shown. It will be apparent to those skilled in the art that various changes may be made without departing from the scope of the invention and the invention is not to be considered limited to what is shown in the drawings and described in the specification. Several features are described hereinafter that can each be used independently of one another or with any combination of other features. However, an individual feature might only address one of the problems discussed above. Some of the problems discussed above might not be fully addressed by any of the features described herein.

What is claimed is:

1. A stabilization medium for extending temporal viability of human semen, the stabilization medium comprising:
one or more pH buffer agents;
one or more inorganic salts;
one or more organic compounds; and
one or more amino acids with antioxidant properties, thereby enabling preservation of semen viability for up to a predetermined time period when the human semen is mixed with the stabilization medium and maintained within a predetermined temperature range.

2. The stabilization medium of claim 1, wherein each amino acid of the one or more amino acids in the stabilization medium has a concentration in a range of 0.01 to 0.5 millimoles (mmol).

3. The stabilization medium of claim 1, wherein the one or more amino acids are selected from a group comprising Alanine (ala), Aspartate (asp), Asparagine (asn), Glutamate (glu), Alanyl-Glutamine (ala-gln), Glycine (gly), Proline (pro) and Serine (ser).

4. The stabilization medium of claim 1, wherein the one or more pH buffer agents comprise 3-(N-morpholino)propanesulfonic acid (MOPS) or 4-(2-Hydroxyethyl)-1-piperazine ethanesulfonic acid (HEPES) in a concentration range of 10.0 to 25.0 millimoles (mmol).

5. The stabilization medium of claim 1, wherein the one or more inorganic salts are selected from a group comprising sodium chloride (NaCl), potassium chloride (KCl), magnesium sulfate heptahydrate (MgSO₄7H₂O), sodium phosphate dihydrate (Na₂PO₄2H₂O), sodium bicarbonate (NaHCO₃), and calcium chloride dihydrate (CaCl₂ 2H₂O).

6. The stabilization medium of claim 1, wherein the one or more inorganic salts comprise calcium chloride dihydrate (CaCl₂.2H₂O) in a concentration between 0.8 to 2.8 millimoles (mmol).

7. The stabilization medium of claim 1, wherein the one or more inorganic salts comprise magnesium sulfate heptahydrate (MgSO₄7H₂O) in a concentration between 0.2 to 4 millimoles (mmol).

8. The stabilization medium of claim 1, wherein the one or more inorganic salts comprise sodium chloride (NaCl) in a concentration between 75.0-105 millimoles (mmol).

9. The stabilization medium of claim 1, wherein the one or more inorganic salts comprise potassium chloride (KCl) in a concentration between 3.5-7.5 millimoles (mmol).

10. The stabilization medium of claim 1, wherein the one or more inorganic salts comprise sodium phosphate dihydrate (Na₂PO₄2H₂O) in a concentration between 0.05-1.5 millimoles (mmol).

11. The stabilization medium of claim 1, wherein the one or more inorganic salts comprise sodium bicarbonate (NaHCO₃) in a concentration between 2.0-10.0 millimoles (mmol).

12. The stabilization medium of claim 1, wherein the one or more organic compounds are selected from a group comprising glucose, sodium lactate (L-isomer), sodium pyruvate and Taurine (tau).

13. The stabilization medium of claim 1, wherein the one or more organic compounds comprise Taurine (tau) in a concentration range of 0.01 to 10.0 millimoles (mmol).

14. The stabilization medium of claim 1, wherein the one or more organic compounds comprise glucose in a concentration between 0.05 to 5.0 millimoles (mmol).

15. The stabilization medium of claim 1, wherein the one or more organic compounds comprise sodium lactate (L-isomer) in a concentration between 5.0 to 20.0 millimoles (mmol).

16. The stabilization medium of claim 1, wherein the one or more organic compounds comprise sodium pyruvate in a concentration between 0.1 to 1.0 millimoles (mmol).

17. The stabilization medium of claim 1, further comprising at least one of the following components that comprises one or more antioxidants, one or more pH-altering compounds, one or more antibiotics, and chelating agents, wherein the one or more antioxidants are selected from a group comprising Acetyl-L-Carnitine, Alpha-Lipoic Acid, and N-Acetyl-L-Cysteine, wherein the one or more pH-altering compounds are selected from a group comprising sodium dihydrogen phosphate dihydrate (NaH₂PO₄2H₂O), magnesium sulfate heptahydrate (MgSO₄7H₂O), calcium chloride dihydrate (CaCl₂2H₂O), sodium lactate (L-isomer) and sodium pyruvate.

18. The stabilization medium of claim 1, wherein the stabilization medium has a pH between 7.2 and 8.

19. The stabilization medium of claim 1, wherein the predetermined time period is up to 72 hours and the predetermined temperature range is 36.1 to 37.2 degree Celsius.

20. A method for extending temporal viability of human semen, the method comprising:
mixing the human semen with the stabilization medium of claim 1 to form a mixture, and
maintaining the mixture of the human semen and the stabilization medium within a temperature range of 36.1 to 37.2 degree Celsius, thereby enabling preservation of semen viability for an extended period of up to 72 hours.

21. A kit comprising the stabilization medium of claim 1.
